(19) United States
(12) Patent Application Publication
(10) Pub. No.: US 2010/0003701 A1
(43) Pub. Date: Jan. 7, 2010

(54) COVALENT MODIFICATION AND CONJUGATION OF LUCIFERASE

(75) Inventors: Amer Alagic, Toronto (CA); Pavel Zhelev, Toronto (CA); Yahia A. Gawad, Mississauga (CA)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: CardioGenics Inc., Mississauga (CA)

(21) Appl. No.: 12/534,250

(22) Filed: Aug. 3, 2009

Related U.S. Application Data
(63) Continuation of application No. 11/411,553, filed on Apr. 26, 2006, now abandoned.

(51) Int. Cl. G01N 33/53; C12N 9/96; C12N 9/02

(52) U.S. Cl. 435/7.8; 435/188; 435/189

(57) ABSTRACT

A process for reversible chemical modification of the luciferase, a process for the covalent conjugation of a reversibly modified luciferase to a chemical moiety (a protein or a binding partner such as biotin or an antibody), a process for reactivation of the reversibly-modified and inactivated luciferase, a process for making the said luciferase conjugates and a bioluminescent assay method that uses covalently conjugated firefly luciferase are taught. The present invention also relates to a composition comprising a reversibly modified luciferase, as well as a composition comprising a reversibly modified luciferase covalently conjugated to a chemical moiety.

### FIELD OF THE INVENTION

The present invention relates to covalently conjugated luciferase, kits, methods of manufacture, and use of same.

### BACKGROUND OF THE INVENTION

Bioluminescence is a widely employed reporting mechanism in both clinical laboratory and biomedical research. Firefly luciferase belongs to a family of luciferin-4 monooxygenase enzymes; it catalyzes the oxidation of luciferin substrates such as D-luciferin (in the presence of Mg-ATP and molecular oxygen) to generate oxyluciferin and light. The enzyme has the highest quantum efficiency of any known bioluminescent reaction with a quantum yield of 0.9. This characteristic makes the enzyme highly desirable for use in assays where enhanced sensitivity is required since bioluminescent-based assays are often highly sensitive and amenable to high-throughput format. Other luciferases are also known in the art and have similar structures and enzymatic activities.

Luciferase has been widely used as a reporter enzyme for various in vivo processes. For example, the luciferase gene can be co-inserted into an organism with a gene of interest. Upon successful transfection, the expression of the target gene then can be monitored by a bioluminescent assay (visualizing or measuring generated light) in the presence of a luciferin substrate. Assays of this type have been used widely in cell and developmental biology. Luciferase can also be used to monitor microbial growth as microorganisms generate ATP, an essential co-factor in luciferase light generating reactions, since there is a well established linear relationship between the quantity of ATP and the amount of light emitted by the luciferase.

Constructs of biological moieties of interest can be generated by chemical means, specifically by covalent chemical cross-linking or conjugation. This approach is beneficial since bioconjugation techniques are well developed and conjugates can be generated quickly and efficiently. Chemical constructs of proteins are achieved by linking entities to protein utilizing chemistries of the active surface groups of a protein. The amine containing side chains of lysine, arginine and histidine typically are exposed on the surface of proteins and can be derivatized with ease for linking moieties to the protein (G. T. Hermanson, Bioconjugation techniques, Academic press, 1996).

The surface amino acid groups of the luciferase are important for luciferin substrate binding, and catalysis, and therefore important in generation of light output. Studies have shown that lysine and cysteine residues are important for luciferase activity (Photochem. Photobiol. 1998, 68 (5), 749-753; Biochem. Biophys. Res. Commun., 2000, 267 (1), 394-397). For example, Lee and co-workers have shown that chemical modification of luciferase with the adenine nucleotide analogue p-fluorosulfonylbenzoyl-5′-adenosine (FSBA) reduced 90% of the enzyme activity by modifying an important lysine residue (Biochemistry, 1981, 20 (5), 1253-1256). Several publications disclosed the loss of light generating activity of luciferase by a significant degree upon selective modification of the luciferase enzyme.

Unfortunately, conjugation reactions commonly employ chemical modification of these same surface residues such as lysine and/or cysteine.

Direct chemical modifications (such as conjugation) of the firefly luciferase surface amino groups have previously been attempted. Frustratingly, since both the conjugation reactions and the enzyme activity (and therefore light output) employ many of the same active surface groups, the enzymatic activity, and, as a result, light output efficiency of the luciferase enzyme was significantly impaired.

Chemical modification of the luciferase enzyme while maintaining some of the enzyme's light generating activity by protection mechanisms has been attempted. These attempts have largely been unsuccessful. The loss of luciferase activity during chemical modification reactions was lessened when luciferase substrate analogues or additives were used as protecting agents. Bacterial luciferase has been shown to be partially protected from inactivation by 2,4-dinitrofluorobenzene (FDNB) by binding long-chain aldehydes or FMN to the enzyme (Photochem. Photobiol. 1989, 50 (6), 817-825). However, this approach has thus far resulted in luciferase with diminished enzymatic activity, and therefore has had limited success in generating functional luciferase conjugates.

U.S. Pat. No. 5,837,465 discloses a method for conjugating luciferase in the presence of ATP and/or D-luciferin substrate. The method involves incubating firefly luciferase with one or more of ATP, magnesium ions and D-luciferin in absence of oxygen at concentrations that allow for protection of luciferase against inactivation during conjugation process to an agent such as an antibody, antigen or a nucleic acid. The residual light activity of such a modified luciferase suffered greatly, and was found to be between 11 to 40% of that of the native enzyme.

Genetic engineering has also been utilized to generate a fusion construct of luciferase and a biotin-binding peptide, for facilitating conjugation to biotin (U.S. Pat. No. 5,843,746 and U.S. Pat. No. 5,814,465). This luciferase-biotin-binding protein construct was successfully used in a bioluminescent-based assay (Lett. Appl. Microbiol. 2005, 41 (5), 379-384) and this luciferase construct is commercially available. However, employing genetic engineering to generate luciferase constructs is a complex and lengthy process and requires modification of the luciferase in a manner that is conjugation-partner specific. Furthermore, such genetic engineering methods can only be used for conjugation partners with a known binding affinity to a known protein sequence to be incorporated into the luciferase protein; such binding sites may also hinder binding of the conjugation partner to the substrate to be analyzed. Finally, such constructs do not provide reversible activation/inactivation of the luciferase.

Methylmaleic anhydrides (methylmaleic anhydride and 2,3-dimethylmaleic anhydride) are known to react with the side chain amino group of lysine residue or with the amino group of an N-terminal amino acid of proteins, at neutral or alkaline pH. The use of 2,3-dimethylmaleic anhydride for reversible modification of amino acids of proteins is known, and widely employed in protein functional studies. This reaction is reversible and lowering the pH of the medium to acidic value regenerates free amino group and methylmaleate or 2,3-dimethylmaleate. If selective modification of amino group is specifically desired, the use of 2,3-dimethylmaleic anhydride is preferred over methylmaleic anhydride since the latter can modify thiol groups of cysteine residues in addition to amino groups (Biochem. Cell Biol., 1989, 67 (1):63-66).

Selective modification of surface amino groups of proteins prior to their conjugation is also known. For example, modification of interleukin 6 (IL-6) with 2,3-dimethylmaleic anhydride prior to conjugation with PEG limited the number of groups that could be effectively modified by conjugation with PEG and therefore protected the activity of IL-6 and generated conjugate that showed 140% increase in specific activity compared to IL-6 that was not pre-treated with 2,3-dimethylmaleic anhydride (Br. J. Haematol. 2001, 112 (1), 181-188). 2,3-dimethylmaleic anhydride was used to modify an antibody for the purpose of retaining its antigen-binding activity. Such a chemically modified antibody was then conjugated to methotrexate, to give an antibody conjugate with retained antigen-biding activity upon removal of 2,3-dimethylmaleyl groups (J. Immunol. Methods, 1987, 104 (1-2), 253-258). It was also documented that modification of D-3-hydroxybutyrate dehydrogenase with 2,3-dimethylmalic anhydride protected the enzyme against inactivation by an inhibitor (Biochem. Cell Biol. 1986, 64 (5), 434-440). Antibodies can also be protected from inactivation by chemical reagents by using 2,3-dimethylmaleic anhydride (J. Chromatogr. 1990, 510, 303-309).

Since some of the surface amino groups of luciferase are involved in substrate binding and/or catalysis, it was previously thought that their employment in conjugation would result in loss of the enzyme light generating activities. Further, it was previously thought that blocking of the surface amino groups prior to conjugation (in order to protect the light emitting activity of the enzyme) would result in conjugation failure or very low conjugation efficiency when conjugation is attempted through amino groups. Such process was therefore thought to have a limited practical utility.

### SUMMARY OF THE INVENTION

One aspect of the present invention is a method for covalent conjugation of luciferase with a chemical moiety comprising (a) reacting a luciferase with a reversible amino acid blocking or modifying agent to form an amino acid-modified luciferase, (b) covalently coupling the amino acid-modified luciferase to the chemical moiety to form a covalently coupled, amino acid-modified luciferase, (c) then reversing the reaction of step (a) to form a covalently coupled luciferase.

In one embodiment, the reversible amino acid blocking or modifying agent is (i) methylmaleic anhydride, (ii) 2,3-dimethylmaleic anhydride, (iii) dansylaminomethylmaleic anhydride, (iv) exo-cis-3,6-endo-epoxy-4,5-cis-epoxyhexahydrophthalic anhydride, (v) 3,4,5,6-tetrahydrophthalic anhydride, (vi) ninhydrin, or (vii) 2-aminothiophenol.

In a further embodiment, wherein the chemical moiety is biotin, an antibody, a modified antibody, an antibody fragment, or a modified antibody fragment.

In a further embodiment, the method comprises an additional step between step (a) and step (b), wherein such additional step comprises reacting the amino acid-modified luciferase with an amino acid blocking or modifying agent wherein such amino acid modifying agent modifies at least one amino acid not previously modified by the reversible amino acid modifying agent.

In a further embodiment, the amino acid modifying agent is a covalent coupling agent.

In a further embodiment, the amino acid blocking or modifying agent is 2-iminothiolane, 1-ethyl-3-(3-dimethylaminopropyl)carbodiimide (EDC), N-hydroxysuccinimide (NHS) N-Hydroxysulfosuccinimide (Sulfo-NHS), Sulfosuccinimidyl acetate (Sulfo-NHS-Acetate), EZ-Link® Sulfo-NHS-Biotin, EZ-Link® Sulfo-NHS-LC-Biotin, EZ-Link® Sulfo-NHS-LC-LC-Biotin, EZ-Link® Sulfo-NHS-SS-Biotin, EZ-Link® NHS-PEO₄-Biotin, EZ-Link® NHS-Biotin, EZ-Link® NHS-LC-Biotin, EZ-Link® NHS-LC-LC-Biotin, EZ-Link® PFP-Biotin, EZ-Link® TFP-PEO-Biotin, EZ-Link® NHS-Iminobiotin-Trifluoroacetimaide, EZ-Link® Biotin-BMCC, EZ-Link® PEO-Iodoacetyl Biotin, EZ-Link® Iodoacetyl-LC-Biotin, EZ-Link® Biotinm-HPDP, EZ-Link® 5-(Biotinamido)pentylamine, EZ-Link® Biotin PEO-Amine, EZ-Link® Biotin-PEO-LC-Amine, EZ-Link® Biocytin-Hydrizide, EZ-Link® Biotin Hydrizide, EZ-Link® Biotin-LC-Hydrizide, EZ-Link® Psoralen-PEO-Biotin, EZ-Link® Photoactivatable Biotin, EZ-Link® Biotin-LC-ASA, EZ-Link® Biocytin, Bolton-Hunter reagent, Sufo-Bolton-Hunter reagent, Succinimidyl 4-hydrizinoic-otinate acetone hydrozone (SANH), C6-Succinimidyl 4-hydrazinoniconitate acetone hydrazone (C6-SANH), Succinimidyl 4-hydrazidoterephthalate hydrochloride (SHTH), Succinimidyl 4-formylbenzoate (SFB), C6-Succinimidyl 4-formylbenzoate (C6-SFB), N-5-Azido-2-nitrobenzoyloxysuccinimide (ANB-NOS), N-[4-(p-azidosalicylamido)butyl-3′-(2-pyridylthio)propionamide (APDP), p-Azidophenyl glyoxal monohydrate (APG), 4-(p-azidosaliclyamido)butilamine (ASBA), Bis-[β-(azidosalicylamido)ethyl]disulfide (BASED), 1,4-bis-maleimidobutane (BMB), 1,4-bis-maleimidyl-2,3-dihydroxybutane (BMDB), Bis-maleimidohexane (BMH), Bis-maleimidoethane (BMOE), N-β-Maleimidopropionic acid (BMPA), N-(β-Maleimidopropionic acid)hydrizide trifluoroacetic acid (BMPH), N-(β-Maleimidopropyloxy)succinimde ester (BMPS), 1,8-Bis-maleimidotriethyleneglycol (BMPEO₃), 1,11-Bis-maleimidotetraethylglycol (BMPEO₄), Bis[2-(succinimidyloxycarbonyloxy)ethyl]sulfone (BSOCOES), Bis(sulfosuccinimidyl)suberate, Dicyclohexylcarbodiimide (DCC), Dimethyl 3,3′-dithiobispropionimidate (DTBP), 3,3′-Dithiobis(sulfosuccinimidyl propionate) (DTSSP), 1,5-Diflouoro-2,4-dinitrobenzene (DFDNB), Dimethyl adipimidate hydrochloride salt (DMA), Dimethylpimelimidate hydrochloride salt (DMP), Dimethyl suberimate hydrochloride salt (DMS), 1,4-Di-[3′-(2′-pyridyldithio)propionamido]butane (DPDPB), Disuccinimidyl lutarate (DSg), Dithiobis(succinimidyl propionate) (DSP), Disuccinimidyl suberate (DSS), Disuccinimidyl tartrate (DST), Dithio-bis-maleimidomethane (DTME), Ethylene glycol bis(succinimidyl succinate) (EGS), N-ε-Maleimidocaproic acid (EMCA), N-ε-maleimidocaproic acid hydrizide (EMCH), N-(ε-Maleimidocarpyloxy)succinimide ester (EMCS), N-(γ-Maleimidobuturyloxy)succinimide ester (GMBS), 1,6-Hexane-bis-vinylsulfone (HBVS), N-κ-Maleimidoundecanoic acid (KMUA), N-κ-Maleimidoundecanoic acid hydrizide (KMUH), Succinimidyl-4-(N-maleimidomethyl)cyclohexane-1-carboxy(6-amidocaproate) (LC-SMCC), Succinimidyl 6-[3-(2-pyridyldithio)propionamido]hexanoate (LC-SPDP), m-Melaimidobenzoyl-Nhydroxy succinimide (MBS), 4-(4-N-maleimidophenyl)butyric acid (MPBH), Methyl N-succinimidyl adipate (MSA), N-hyroxysuccinimidyl-4-azidosalicylic acid (NHS-ASA), 3-(2-Pyridyldithio)propionul hydrazide (PDPH), N-(p-maleimidophenyl)isocyanate (PMPI), N-succinimidyl (4-azidophenyl) 1,3′-dithiopropionate (SADP), sulfosuccinimidyl 2-(7-azido-4-methyl-coumarin-3-acetamido)ethyl-1,3′-dithiopropionate (SAED), Sulfosuccinimidyl 2-(m-azido-o-nitrobenzamido)ethyl-1,3′-dithiopropionate (SAND), N-Succinimidyl 6-(4′-azido-2′-nitrophenylamino)hexanoate (SANPAH), Sulfosuccinimidyl-2-(-p-azido-salicylamido)ethyl-1,3′-dithiopropionate (SASD), N-Succinimidyl S-acetylthioacetate (SATA), N-succinimidyl S-acetylthiopropionate (SATP), Succinimidyl 3-(bromoacetamido)propionate (SBAP), Sulfosuccinimidyl-(perfluoroazidobenzamido)ethyl-1,3′-dithiopropionate (SFAD), N-Succinimidyl iodoacetate (SIA), N-Succinimidyl(4-iodoacetyl)aminobenzoate (SIAB), Succinimidyl 4-(N-maleimidomethyl)cyclohexane-1-carboxylate (SMCC), Succinimidyl 4-(p-maleimidophenyl)butyrate (SMPB), Succinimidyl-6-(β-maleimidopropionamido)hexanoate (SMPH), 4-Succinimidyloxycarbonyl-methyl-α-(2-propyldithio)toluene (SMPT), Succinimidyl-[4-(psoralen-8-yloxy)butyrate (SPB), N-Succinimidyl 3-(2-pyridylthio)propionate (SPDP), Disulfosuccinimidyl tartrate (Sulfo-DST), Ethylene glycol bis(sulfosuccinimidyl succinate) (Sulfo-EGS), N-(ε-Maleimidocapryloxy)sulfosuccinimide ester (Sulfo-EMCS), N-(γ-Maleimidobuturyloxy)sulfosuccinimide ester (Sulfo-GMBS), N-Hydroxysulfosuccinimidyl-4-azidobenzoate (Sulfo-HSAB), N-(κ-Maleimidoundecanoyloxy)sulfosuccinimide ester (Sulfo-KMUS), Sulfosuccinimidyl-6-[α-methyl-α-(2-pyridyldithio)toluamido]hexanoate (Sulfo-LC-SMPT), Sulfosuccinimidyl 6-[3′-(2-pyridyldithio)propionamido]hexanoate (Sulfo-LC-SPDP), m-Maleimidobenzoyl-N-hydroxysulfosuccinimide (Sulfo-MBS), Sulfosuccinimidyl(4-azidosalicylamido)hexanoate (Sulfo-NHS-LC-ASA), Sulfosuccinimidyl(4-azidophenyldithio)propionate (Sulfo-SADP), Sulfo-succinimidyl 6-(4′-azido-2′-nitro-phenylamino)hexanoate (Sulfo-SANPAH), Sulfosuccinimidyl(4-iodoacetyl)aminobenzoate (Sulfo-SIAB), Sulfosuccinimidyl 4-(N-maleimidomethyl)cyclohexane-1-carboxylate (Sulfo-SMCC), Sulfosuccinimidyl 4-(p-maleimidophenyl)butyrate (Sulfo-SMPB), N-(ε-Trifluoroacetylcapryloxy)succinimide ester (TFCS), Sulfosuccinimidyl[2-6-(biotinamido)-2-(p-azidobenzamido)-hexanoamido[ethyl-1,3′-dithiopropionate (Sulfo-SBED), Succinimidyl[2-6-(biotinamido)-2-(p-azidobenzamido)-hexanoamido[ethyl-1,3′-dithiopropionate (SBED), Tris-succinimidyl aminotriacetate (TSAT), β-[Tris(hydroxymethyl)phosphino]propionic acid (THPP), Tris(2-maleimidomethyl)amine (TMEA), N-(Iodoethyl)trifluoroacetamide, 2-Aminoethyl-2′-aminothanethiosulfonate, 5,5′-Dithio-bis(2-nitrobenzoic acid), N-Ethylmaleimide, p-Hydroxyphenylglyoxal, Iodoacetic acid, Phenylmethylsulfonyl fluoride (PMSF), 1-Fluoro-2,4-dinitrophenyl-5-L-alenine amide (FDAA), Fluoraldehyde o-Phthaldehyde, Ninhydrin, Phenylisothiocyanate, Ammonium 4-chloro-7-sulfobenzo-furzan (SBF-Chloride), 2,4,6-Trinitrobenzene sulfonic acid, or Methylmethanothiosulfonate (MMTS).

In a further embodiment, the method further comprises (1) a blocking step prior to step (a), wherein such blocking step comprises reacting the luciferase with a luciferin substrate or an Mg-ATP complex substrate; and (2) a washing step prior to, during or after step (c) wherein such washing step removes the luciferin substrate or the Mg-ATP complex substrate.

In a further embodiment, step (a) occurs at a pH of between 5 and 9.

In a further embodiment, step (a) occurs for between 10 minutes and 24 hours, for example, for between 30 and 60 minutes.

In a further embodiment, the ratio of reversible amino acid modifying agent to luciferase in step (a) is 80 to 100 molar equivalents of reversible amino acid modifying agent to luciferase.

In a further embodiment, the ratio of chemical moiety to amino acid-modified luciferase in step (b) is between 0.2-10 molar equivalents of chemical moiety to amino acid-modified luciferase.

In a further embodiment, step (b) occurs at a pH of between 6 and 9.

In a further embodiment, the covalent coupling in step (b) is done by mixing.

In a further embodiment, the ratio of amino acid modifying agent to amino acid-modified luciferase in the additional step is about 100 molar equivalents of amino acid modifying agent to amino acid-modified luciferase.

In a further embodiment, the reversal step (c) comprises hydrolysis of the amino acid-modified luciferase.

In a further embodiment, steps (b) and (c) occur simultaneously.

In yet a further embodiment, the reversal step (c) occurs at a pH of about 6.

Another aspect of the present invention is a method for performing a binding assay comprising use of a covalently conjugated luciferase made by a method as described herein.

Another aspect of the present invention is a method for performing a binding assay to determine a concentration or a presence of a substrate in a sample, said method comprising the steps of (a) mixing the sample with a luciferase covalently conjugated to a chemical moiety known to have a specific binding activity for the substrate, so that a substrate-luciferase-chemical entity complex is formed, (b) separating said substrate-luciferase-chemical entity complex from any free covalently conjugated luciferase, (c) adding a signal generating system that generates a signal when enzymatically modified by luciferase, and (d) measuring the signal.

Another aspect of the present invention is a method for performing a binding assay to determine a concentration or a presence of a substrate in a sample, said method comprising the steps of (a) mixing said sample with a first binding partner, said first binding partner having a known affinity to said substrate, said first binding partner also having a known affinity to a chemical moiety, to form a substrate-first binding partner complex, (b) contacting said substrate-first binding partner complex with a luciferase covalently bound to said chemical moiety to form a tertiary complex of substrate-first binding partner-luciferase covalently bound to chemical moiety, (c) separating said tertiary complex from remaining luciferase covalently bound to said chemical moiety, (d) adding a signal generating system that generates a signal when enzymatically modified by luciferase, and (e) measuring said signal.

In one embodiment of the present invention, the luciferase covalently conjugated to a chemical moiety is made by a method described herein.

In one aspect of the present invention, the chemical moiety is biotin, an antibody, or an antibody fragment.

One aspect of the present invention is luciferase covalently conjugated to a chemical moiety and having luciferase enzyme activity similar to a non covalently conjugated luciferase.

In one embodiment, the luciferase covalently conjugated to a chemical moiety and having luciferase enzyme activity similar to a non covalently conjugated luciferase has a chemical moiety selected from biotin, an antibody, an antibody fragment, a modified antibody, or a modified antibody fragment.

A further aspect of the present invention is the use of the luciferase covalently conjugated to a chemical moiety as described herein in an immunoassay.

A further aspect of the present invention is a kit comprising (a) the luciferase covalently conjugated to a chemical moiety as described herein, (b) a signal generating system, and (c) instructions for use of (a) and (b) in an immunoassay.

A further aspect of the present invention is use of the luciferase covalently conjugated to a chemical moiety, made by any method described herein, in an immunoassay.

A further aspect of the present invention is a kit comprising (a) a luciferase covalently conjugated to a chemical moiety, made by the method of any one of claims 1-18, (b) a signal generating system, and (c) instructions for use of (a) and (b) in an immunoassay.

In one embodiment, the signal generating system comprises Mg-ATP and luciferin.

In another embodiment, the signal generating system comprises Mg-ATP and luciferin.

Another aspect of the present invention is a reversibly chemically modified luciferase made by a method comprising reacting luciferase with a reversible amino acid modifying agent to form an amino-acid modified luciferase.

### DETAILED DESCRIPTION

The present inventors have devised a method of protecting luciferase's important surface residues by using chemical reagents in order conjugate protected luciferase to a moiety of interest. The protecting agent is then removed in order to obtain a functional luciferase conjugate with high specific activity. Such an approach can be used to generate different, varied functional conjugates of luciferase that could have practical applications in bioluminescent-based assays, including immunoassays. The use of luciferase for immunoassays offer benefits of high sensitivity and extended signal. One of the advantages of this approach is its versatility as it can be utilized to generate conjugates “by design”.

When utilized in this patent specification, the following terms have the following meanings.

The term “antibody” is used in the broadest sense and specifically covers monoclonal antibodies (including full length monoclonal antibodies), polyclonal antibodies, multispecific antibodies (e.g. bispecific antibodies), modified antibodies, antibody fragments, and modified antibody fragments so long as they exhibit the desired biological activity.

Modified antibodies are antibodies that have been modified, either using molecular biology or chemical methodologies. Modified antibodies exhibit the desired biological activity, but have been modified for ease of use, ease of manufacture, or specialized activity. Modified antibodies include, for example, single chain antibodies, chimeric antibodies, and CDR-grafted antibodies, including compounds which include CDR sequences which specifically recognize a specific polypeptide, as well as humanized antibodies.

“Activity fragments” comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparation, which typically include different antibodies directed against different determinants (epitopes), each monoclonal antibody is directed against a single determination on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the technique described in Clackson et al., Nature 352:624-626 (1991) and Marks et al., J. Mol. Biol. 222:581-597 (1991), for example.

The term “hypervariable region” when used herein refers to the amino acid residues of an antibody which are responsible for antigen-binding. The hypervariable region comprises amino acid residues from a “complementarity determining region” or “CDR” (i.e. residues 24-34 (L1), 50-58 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain. Chothia and Lesk J. Mol. Biol. 196:901-917 (1987)). “Framework” or “FR” residues are those variable domain residues other than the hypervariable region residues as herein defined. The CDR and FR residues of the H52 antibody of the example below are identified in Elgenbrot et al. Proteins: Structure, Function and Genetics 18:49-62 (1994).

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

“Single-chain Fv” or “sFv” antibody fragments comprise the V_{H} and V_{L} domain of antibody, wherein these domains are present in a single polypeptide chain. Generally, the Fv polypeptide further comprises a polypeptide linker between the V_{N} and V_{L} domains which enables the SFv to form the desired structure for antigen binding. For a view of sFv see Pluckthun in The Pharmacology of Monoclonal Antibodies, vol. 113, Rosenburg and Moore eds. Springer-Verlag, N.Y., pp. 269-315 (1994).

The term “diabodies” refers to small antibody fragments with two antigen-binding sites, which fragments comprise a heavy chain variable domain (V_{M}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

The expression “linear antibodies” when used throughout the application refers to the antibodies described in Zapata et al. Protein Eng. 8 (10):1057-1062 (1995). Briefly, these antibodies comprise a pair of tandem Fd segments (V_{H}-C_{H}1-V_{H}-C_{N}1) which form a pair of antigen binding regions. Linear antibodies can be bispecific or monospecific.

The expression “amino acid blocking or modifying agent” refers to any chemical or biological agent that is able to bind to, modify, or hinder the activity of an amino acid that is part of a polypeptide or protein. An amino acid blocking or modifying agent can, for example, bind to a specific residue, such as a lysine residue and cause inactivity of that residue, or of that activity region of the protein. Examples of amino acid blocking or modifying agents include 2-iminothiolane, 1-ethyl-3-(3-dimethylaminopropyl)carbodiimide (EDC), N-hydroxysuccinimide (NHS) N-Hydroxysulfosuccinimide (Sulfo-NHS), Sulfosuccinimidyl acetate (Sulfo-NHS-Acetate), EZ-Link® Sulfo-NHS-Biotin, EZ-Link® Sulfo-NHS-LC-Biotin, EZ-Link® Sulfo-NHS-LC-LC-Biotin, EZ-Link® Sulfo-NHS-SS-Biotin, EZ-Link® NHS-PEO₄-Biotin, EZ-Link® NHS-Biotin, EZ-Link® NHS-LC-Biotin, EZ-Link® NHS-LC-LC-Biotin, EZ-Link® PFP-Biotin, EZ-Link® TFP-PEO-Biotin, EZ-Link® NHS-Iminobiotin-Trifluoroacetimaide, EZ-Link® Biotin-BMCC, EZ-Link® PEO-Iodoacetyl Biotin, EZ-Link® Iodoacetyl-LC-Biotin, EZ-Link® Biotinm-HPDP, EZ-Link® 5-(Biotinamido)pentylamine, EZ-Link® Biotin PEO-Amine, EZ-Link® Biotin-PEO-LC-Amine, EZ-Link® Biocytin-Hydrizide, EZ-Link® Biotin Hydrizide, EZ-Link® Biotin-LC-Hydrizide, EZ-Link® Psoralen-PEO-Biotin, EZ-Link® Photoactivatable Biotin, EZ-Link® Biotin-LC-ASA, EZ-Link® Biocytin, Bolton-Hunter reagent, Sufo-Bolton-Hunter reagent, Succinimidyl 4-hydrizinoic-otinate acetone hydrozone (SANH), C6-Succinimidyl 4-hydrazinoniconitate acetone hydrazone (C6-SANH), Succinimidyl 4-hydrazidoterephthalate hydrochloride (SHTH), Succinimidyl 4-formylbenzoate (SFB), C6-Succinimidyl 4-formylbenzoate (C6-SFB), N-5-Azido-2-nitrobenzoyloxysuccinimide (ANB-NOS), N-[4-(p-azidosalicylamido)BUTYL-3′-(2-pyridylthio)propionamide (APDP), p-Azidophenyl glyoxal monohydrate (APG), 4-(p-azidosaliclyamido)butilamine (ASBA), Bis-[β-(azidosalicylamido)ethyl]disulfide (BASED), 1,4-bis-maleimidobutane (BMB), 1,4-bis-maleimidyl-2,3-dihydroxybutane (BMDB), Bis-maleimidohexane (BMH), Bis-maleimidoethane (BMOE), N-β-Maleimidopropionic acid (BMPA), N-(β-Maleimidopropionic acid)hydrizide trifluoroacetic acid (BMPH), N-(β-Maleimidopropyloxy)succinimde ester (BMPS), 1,8-Bis-maleimidotriethyleneglycol (BMPEO₃), 1,11-Bis-maleimidotetraethylglycol (BMPEO₄), Bis[2-(succinimidyloxycarbonyloxy)ethyl]sulfone (BSOCOES), Bis(sulfosuccinimidyl)suberate, Dicyclohexylcarbodiimide (DCC), Dimethyl 3,3′-dithiobispropionimidate (DTBP), 3,3′-Dithiobis(sulfosuccinimidyl propionate) (DTSSP), 1,5-Diflouoro-2,4-dinitrobenzene (DFDNB), Dimethyl adipimidate hydrochloride salt (DMA), Dimethylpimelimidate hydrochloride salt (DMP), Dimethyl suberimate hydrochloride salt (DMS), 1,4-Di-[3′-(2′-pyridyldithio)propionamido]butane (DPDPB), Disuccinimidyl lutarate (DSg), Dithiobis(succinimidyl propionate) (DSP), Disuccinimidyl suberate (DSS), Disuccinimidyl tartrate (DST), Dithio-bis-maleimidomethane (DTME), Ethylene glycol bis(succinimidyl succinate) (EGS), N-ε-Maleimidocaproic acid (EMCA), N-ε-maleimidocaproic acid hydrizide (EMCH), N-(ε-Maleimidocarpyloxy)succinimide ester (EMCS), N-(γ-Maleimidobuturyloxy) succinimide ester (GMBS), 1,6-Hexane-bis-vinylsulfone (HBVS), N-κ-Maleimidoundecanoic acid (KMUA), N-κ-Maleimidoundecanoic acid hydrizide (KMUH), Succinimidyl-4-(N-maleimidomethyl)cyclohexane-1-carboxy(6-amidocaproate) (LC-SMCC), Succinimidyl 6-[3-(2-pyridyldithio)propionamido]hexanoate (LC-SPDP), m-Melaimidobenzoyl-Nhydroxy succinimide (MBS), 4-(4-N-maleimidophenyl)butyric acid (MPBH), Methyl N-succinimidyl adipate (MSA), N-hyroxysuccinimidyl-4-azidosalicylic acid (NHS-ASA), 3-(2-Pyridyldithio)propionul hydrazide (PDPH), N-(p-maleimidophenyl)isocyanate (PMPI), N-succinimidyl (4-azidophenyl) 1,3′-dithiopropionate (SADP), sulfosuccinimidyl 2-(7-azido-4-methyl-coumarin-3-acetamido)ethyl-1,3′-dithiopropionate (SAED), Sulfosuccinimidyl 2-(m-azido-o-nitrobenzamido)ethyl-1,3′-dithiopropionate (SAND), N-Succinimidyl 6-(4′-azido-2′-nitrophenylamino)hexanoate (SANPAH), Sulfosuccinimidyl-2-(-p-azido-salicylamido)ethyl-1,3′-dithiopropionate (SASD), N-Succinimidyl S-acetylthioacetate (SATA), N-succinimidyl S-acetylthiopropionate (SATP), Succinimidyl 3-(bromoacetamido)propionate (SBAP), Sulfosuccinimidyl-(perfluoroazidobenzamido)ethyl-1,3′-dithiopropionate (SFAD), N-Succinimidyl iodoacetate (SIA), N-Succinimidyl(4-iodoacetyl)aminobenzoate (SIAB), Succinimidyl 4-(N-maleimidomethyl)cyclohexane-1-carboxylate (SMCC), Succinimidyl 4-(p-maleimidophenyl)butyrate (SMPB), Succinimidyl-6-(β-maleimidopropionamido)hexanoate (SMPH), 4-Succinimidyloxycarbonyl-methyl-α-(2-propyldithio)toluene (SMPT), Succinimidyl-[4-(psoralen-8-yloxy)butyrate (SPB), N-Succinimidyl 3-(2-pyridylthio)propionate (SPDP), Disulfosuccinimidyl tartrate (Sulfo-DST), Ethylene glycol bis(sulfosuccinimidyl succinate) (Sulfo-EGS), N-(ε-Maleimidocapryloxy)sulfosuccinimide ester (Sulfo-EMCS), N-(γ-Maleimidobuturyloxy)sulfosuccinimide ester (Sulfo-GMBS), N-Hydroxysulfosuccinimidyl-4-azidobenzoate (Sulfo-HSAB), N-(κ-Maleimidoundecanoyloxy)sulfosuccinimide ester (Sulfo-KMUS), Sulfosuccinimidyl-6-[α-methyl-α-(2-pyridyldithio)toluamido]hexanoate (Sulfo-LC-SMPT), Sulfosuccinimidyl 6-[3′-(2-pyridyldithio)propionamido]hexanoate (Sulfo-LC-SPDP), m-Maleimidobenzoyl-N-hydroxysulfosuccinimide (Sulfo-MBS), Sulfosuccinimidyl(4-azidosalicylamido)hexanoate (Sulfo-NHS-LC-ASA), Sulfosuccinimidyl(4-azidophenyldithio)propionate (Sulfo-SADP), Sulfo-succinimidyl 6-(4′-azido-2′-nitro-phenylamino)hexanoate (Sulfo-SANPAH), Sulfosuccinimidyl(4-iodoacetyl)aminobenzoate (Sulfo-SIAB), Sulfosuccinimidyl 4-(N-maleimidomethyl)cyclohexane-1-carboxylate (Sulfo-SMCC), Sulfosuccinimidyl 4-(p-maleimidophenyl)butyrate (Sulfo-SMPB), N-(ε-Trifluoroacetylcapryloxy)succinimide ester (TFCS), Sulfosuccinimidyl[2-6-(biotinamido)-2-(p-azidobenzamido)-hexanoamido[ethyl-1,3′-dithiopropionate (Sulfo-SBED), Succinimidyl[2-6-(biotinamido)-2-(p-azidobenzamido)-hexanoamido[ethyl-1,3′-dithiopropionate (SBED), Tris-succinimidyl aminotriacetate (TSAT), β-[Tris(hydroxymethyl)phosphino]propionic acid (THPP), Tris(2-maleimidomethyl)amine (TMEA), N-(Iodoethyl)trifluoroacetamide, 2-Aminoethyl-2′-aminothanethiosulfonate, 5,5′-Dithio-bis(2-nitrobenzoic acid), N-Ethylmaleimide, p-Hydroxyphenylglyoxal, Iodoacetic acid, Phenylmethylsulfonyl fluoride (PMSF), 1-Fluoro-2,4-dinitrophenyl-5-L-alenine amide (FDAA), Fluoraldehyde o-Phthaldehyde, Ninhydrin, Phenylisothiocyanate, Ammonium 4-chloro-7-sulfobenzo-furzan (SBF-Chloride), 2,4,6-Trinitrobenzene sulfonic acid, and Methylmethanothiosulfonate (MMTS).

The term “signal generating system” means any compound or set of compounds capable of generating a signal when contacted with a known reactant to the signal generating system. The signal is typically of varying strength depending on the amount of reactant present. For example, a signal generating system for luciferase would comprise Mg-ATP and luciferin, and would generate light upon contact with the reactant luciferase.

The present inventors have discovered a method for the selective utilization of functional groups of the luciferase for conjugation to a moiety without a significant loss of activity of the luciferase. The method comprises steps of selectively and reversibly blocking of amino groups essential for enzyme activity, covalently conjugating a chemical moiety to amino groups of such chemically modified luciferase that are not essential for its enzymatic activity then temporally releasing the blocked groups in order to regain the enzymatic activity of the conjugated luciferase. Such conjugated luciferase can be used in bioluminescent-based assays, including immunoassays. The method is efficient and offers opportunities for generating constructs of luciferase with various chemical and biological entities.

It has now been found that luciferase can be covalently conjugated by chemical means to either a synthetic/organic moiety (such as chemically activated biotin) or a natural/biological moiety (such as an antibody or a protein) (both collectively referred to as a “chemical moiety”) in a manner that results in obtaining luciferase constructs wherein the luciferase enzyme retains more than 90% of its enzymatic light-generating activity in comparison to its native counterpart. In accordance with the invention, instead of protecting the active and/or binding site of the enzyme with the substrate (D-luciferin) or the co-factor (Mg-ATP), or making luciferase constructs by genetic engineering, a totally different method is discovered. The method employs reversible chemical modification of lysine residues that are important for the enzyme's light output, thus inactivating the enzyme, followed by conjugation of such inactive luciferase to a desired moiety, then removing the inactivating groups from the enzyme to generate active and functional luciferase constructs.

Reversible chemical modification and inactivation of luciferase is accomplished by reacting the enzyme with amino-specific blocking or modifying reagent that can be removed under specific reaction conditions. This reagent modifies luciferase's lysine residues that are essential for the enzyme's light output and are accessible and available for the modification reaction due to their spatial arrangements (and hence structural properties) or due to their ionization state (and hence reactivity). Such modifications of luciferase generate the inactivated enzyme.

Upon treatment of luciferase with the amino-specific blocking or modifying reagent, there are lysine residues remaining which are not modified in the process either due to their spatial arrangement or their reactivity. Those remaining unmodified lysine residues can then be used in a conjugation process as points of attachment of a desired moiety to inactivated luciferase. Specific reaction conditions (such as pH, ionic strength, charge distribution of a modified protein, etc) can have an effect on the reactivity of those lysine groups and enable their direct coupling to an amino-specific reagent for the purpose of conjugation or can enable their modification for the purpose of converting an amino group to another chemically reactive group such as, but not limited to, thiol group.

We utilized those remaining amino groups of an inactivated luciferase for direct coupling to a moiety of interest and for their conversion to another chemically reactive group (specifically a thiol group) for the purpose of obtaining covalent luciferase constructs.

Following the conjugation process, the deprotection of essential lysine residue from inactivated conjugated luciferase is effected by specific reaction conditions. This process generates luciferase constructs that retain high enzymatic light generating activity.

We have discovered that the light emitting activities of the luciferase enzyme can be preserved by reversible blocking and protection process of the surface amino groups, while obtaining an effective conjugation of the luciferase enzyme through the remaining amino groups that were not involved in light generating activities. We have invented a new method for generating functional luciferase conjugates that involves reversible protection of luciferase by reversibly modifying it by chemical means for example, utilizing a 2,3-dimethylmaleic anhydride reagent to form a luciferase containing 2,3-dimethymaleyl groups, followed by a conjugation process of the luciferase to a chemical moiety, then removing the 2,3-dimethylmaleyl groups from the conjugated luciferase by hydrolysis.

Optionally, if lysine residues that are essential for luciferase activity due to their involvement in luciferin substrate or Mg-ATP complex substrate binding, or lysine residues proximal to such a substrate binding site, they may be protected from inactivation by incubating luciferase with either or both of its substrates (D-luciferin or Mg-ATP complex) prior to modification by 2,3-dimethylmaleic anhydride. Such process offers additional protection against inactivation during the conjugation process.

We have found that luciferase conjugates of the present invention are useful for conducting a biochemical binding assay for a substrate of interest that may or may not be present in a sample, such binding assay comprising preparing, in a liquid medium, a mixture of the sample with a specific binding partner for said substrate, to form a complex of the substrate and the specific binding partner to the substrate (if the substrate is present in the sample), said mixture also comprising other components of a signal generating system wherein one of the components is a chemically conjugated luciferase as described above, such conjugated luciferase having a chemical moiety that is capable of binding to the specific binding partner, and measuring the signal generated by chemically conjugated luciferase in such system.

“Substrate” is a commonly used term that denotes a target compound whose presence and/or quantity is to be determined in a test medium. The substrate is usually one of the components of a system which involves binding reactions based on bioaffinity or enzyme catalyzed reactions. A specific binding partner known to have specific binding affinity for a substrate under testing, such as antibody, natural binding protein, lectin, enzyme, receptor, DNA, RNA, peptide nucleic acid (PNA), artificial antibody, or nucleic probe is used to form a complex with the substrate, and can include a label to quantify the complex. Luciferase that was chemically conjugated as previously described can be applied to provide to the reaction medium any of the active components required to form a complex of the substrate and binding partner, or to be a part of a signal generating system.

The present invention provides a method for making covalent conjugates by a process in which luciferase is initially inactivated, then conjugated to either an organic or a biological entity, followed by removal of deactivating groups from the conjugated luciferase to obtain functional and active luciferase conjugates.

The present invention also provides a method for a making a reversibly chemically modified luciferase. Functionalizing reagents are used for selective chemical modification to introduce a 2,3-dimethylmaleyl group to the polypeptide chain of luciferase at one or more amino groups contained therein and which are essential for the enzyme's activity. This reagent can react with amino groups of luciferase at neutral and alkaline pH (pH range: 7 to 9). An optimum time for incubation of luciferase with 2,3-dimethylmaleic anhydride is between 30-60 min. Reaction time can be extended up to 24 hours; however, extending the reaction beyond 1 hr did not affect significantly the outcome of the modification reaction.

Firefly luciferase enzyme has 40 lysine residues (Conti, et. al, Structure, 1996, 4, 287-298) that theoretically can be modified with 2,3-dimethylmaleic anhydride. Different molar ratios of this compound to luciferase can be used. A molar range of between 50 to 130, or 70 to 110, or preferably 80 to 100 molar equivalents of 2,3-dimethylmaleic anhydride to luciferase worked well. Reducing the amount of the reagent below 80 molar equivalents can result in obtaining luciferase products wherein some of the essential residues were not modified. Utilization of those un-protected essential residues in further conjugation steps can generate irreversibly inactivated enzyme.

The present invention also provides a method for making a reversibly modified luciferase and the utilization of this reversibly modified luciferase in conjugation to a chemical moiety by utilizing amino acid residues of the enzyme that were not modified by 2,3-dimethylmaleic anhydride.

In cases when further modification of 2,3-dimethylmaleyl luciferase was not required, the conjugation reaction could be performed in the same medium as the 2,3-dimethylmalylation reaction, if the conditions required for the conjugation reaction permit it. Alternatively, the excess of unreacted or hydrolyzed 2,3-dimethylmaleic anhydride can be removed by desalting or by an other known process. We conjugated 2,3-dimethylmaleyl luciferase to NHS-activated biotin with equal success in medium that does and does not contain the excess of unreacted or hydrolyzed 2,3-dimethylmaleic anhydride. We used between 5-10 molar equivalents of NHS-biotin to 2,3-dimethylmaleyl luciferase and found that an optimum amount for conjugation reactions of this type was 10 molar equivalents of NHS-biotin to luciferase at neutral to alkaline pH (pH range from 7-9). If lower amounts of NHS-biotin were used, the reaction mixture containing 2,3-dimethylmaleyl luciferase and NHS-biotin needed to be mixed faster; however, this led to lower recovery of the enzyme due to the precipitation of the enzyme. If the reaction containing NHS-biotin and 2,3-dimethylmaleyl luciferase was not mixed at all, the amount of NHS-biotin needed to be increased.

The invention further comprises a method for making a reversibly modified luciferase and the utilization of this reversibly modified luciferase in reactions that chemically modify the remaining amino acid residues of said luciferase. In some cases, further modification of 2,3-dimethylmaleyl luciferase is required (such as modification with 2-iminothiolane or with any other reagent that converts one type of functional group into another). In these cases, the excess of unreacted or hydrolyzed 2,3-dimethylmaleic anhydride can be removed by desalting or by any other known method. Some or all of the amino groups of 2,3-dimethylmaleyl luciferase that did not react with 2,3-dimethylmaleic anhydride can then be converted to thiol groups by using an amino acid modifying agent such as 2-iminothiolane reagent at a pH of about 7.5. This reaction irreversibly introduced non-native thiol groups into 2,3-dimethylmaleyl luciferase and generated 2-iminothiolyl-2,3-dimethylmaleyl luciferase. For this reaction, we used 100 molar equivalents of 2-iminothiolane reagents to ensure maximum possible conversion of amino groups to thiol groups in order to generate 2-imoniothiolyl 2,3-dimethylmaleyl luciferase. Non-native thiol groups of 2,3-dimethylmaleyl luciferase, which were introduced by the treatment with 2-iminothiolane, were utilized as points of attachments of various entities during conjugation reactions.

The present invention also comprises a method for making a reversibly modified luciferase, the utilization of this reversibly modified luciferase in reactions that chemically modify and activate remaining amino acid residues of said luciferase and the utilization of such reversibly modified and activated luciferase in processes of conjugation to chemical moieties of interest.

2-imoniothiolyl 2,3-dimethylmaleyl luciferase was conjugated to an succinimidyl 4-N-maleimidomethyl cyclohexane-1-carboxylate (SMCC)-activated antibody. The ratio of the luciferase to the SMCC-activated antibody (the chemical moiety) was 5 molar equivalents to 1. Although higher amounts of luciferase could be used, we found that higher amounts of the enzyme did not increase the yield of the conjugation reaction. The conjugation reaction was performed at a pH of about 6. At this pH, the formation of a thio-ether group is believed to occur through the reaction between the non-native thiol group on the modified luciferase and the maleimide group on the SMCC-activated antibody. The formation of a stable covalent thioether bond in such manner irreversibly modified the 2,3-dimethylmaleyl luciferase.

The present invention also includes a method for removal of a moiety from luciferase that is introduced in a process of making a reversibly chemically modified luciferase.

In order to obtain covalent luciferase conjugates of high specific enzymatic activity we used enzyme residues that are important for a substrate/co-factor binding and/or catalysis that were in their native form following chemical manipulations of the enzyme. The modification of luciferase with 2,3-dimethylmaleic anhydride reversibly inactivated the enzyme, by introducing acid-labile 2,3-dimethylmaleyl groups. Optionally, further modification of 2,3-dimethylmaleyl luciferase residues that did not react with 2,3-dimethylmaleic anhydride was accomplished by different chemical reagents, which permanently modified the enzyme to give stable covalent conjugates of 2,3-dimethylmaleyl luciferase. The re-activation of the such conjugated luciferase having high enzymatic light-generating activity was accomplished by hydrolysis of acid-labile 2,3-dimethylmaleyl groups. The hydrolysis reaction occurred during the conjugation reaction, which was performed in acidic medium (pH 6). This method regenerated native lysine residues that are essential for activity on the conjugated luciferase and produced covalent conjugates of luciferase having high specific enzymatic light-generating activity.

We have also developed a method of using these conjugates in bioluminescent-based assays. Also disclosed is a method for quantitatively assaying a fluid for the presence of an unknown quantity of antigen in a system wherein the said conjugated luciferase is employed.

The present invention thus teaches a versatile method for generating different types of luciferase conjugates having high specific enzymatic light-generating activity: biotinylated luciferase and antibody-luciferase conjugate. These two conjugates can have numerous applications in bioluminescent based assays, including immunoassays. Biotinylated luciferase can be utilized in assays as a part of a detecting system that also contains a biotin-binding element such as biotin-binding peptide, avidin, streptavidin, Neutravidin™ and/or any other synthetic or natural entity that has an affinity toward biotin or luciferase (such as anti-luciferase antibodies). Similarly, the antibody-luciferase conjugate can be used in an assay in which the antibody-luciferase conjugate is either a part of an antigen-detecting system or part of a signal-generating system.

The preceding description and following examples are illustrative embodiments of the present invention. It is to be understood that the invention is not limited to these precise embodiments, and that various changes and modifications may be effected therein by one skilled in the art. Some of such changes and modifications are encompassed in the appended claims.

### EXAMPLES

### Example 1

### Biotinylation of Thermostable Firefly Luciferase

### Materials:

Thermostable firefly luciferase (Luc-T) and D-luciferin were obtained from Kikkoman Corporation (Japan). Casein (vitamin free) 2,3-dimethylmaleic anhydride, anhydrous dimethyl sulfoxide and ATP were purchased from Sigma. Streptavidin was obtained from Calbiochem. Sulfo-SMCC (sulfosuccinimidyl 4-N-maleimidomethyl cyclohexane-1-carboxylate), 2-iminothiolane, EZ-Link™ Sulfo-HNS-LC-LC-biotin, EZ™ Biotin quantitation kit and BCA protein assay kit were obtained from Pierce. Bovine fetal serum was obtained from HyClone. Mouse anti-human creatin kinase BB antibody was obtained from DakoCytomation. Troponin T monoclonal (MAK TN-T M11-7) and (MAK TN-T M7) antibodies were obtained from Roche Diganostics. High bind strip well plates-white with flat bottom were obtained from Greiner Bio-One. All other reagents were of molecular biology grade or better.

### Preparation of Luciferase:

Lyophilized luciferase was prepared by dissolving the enzyme in buffer (50 mM phosphate, 0.1 mM EDTA, pH 7.8) to a final enzyme concentration of 5 mg/mL.

### Modification of luciferase with 2,3-dimethylmaleic anhydride (DMMA) and biotinylation

Luciferase (0.25 mg, 4.2×10⁻⁹ mol) was mixed with 0.45 mL 0.01 mM phosphate, 0.14 M NaCl, buffer pH 7.4. DMMA (0.04 mg, 3.4×10⁻⁷ mol) that was prepared in anhydrous DMSO was added to the luciferase solution and the reaction proceeded for sixty minutes at 22° C. with constant, slow mixing. After sixty minutes, NHS-biotin (0.03 mg, 4.2×10⁻⁸ mol) was added to reaction mixture containing luciferase and DMMA. The coupling reaction proceeded for thirty minutes at 22° C.

The sample was desalted on a 5 ml Hi Trap™ column equilibrated with 50 mM MES, 2 mM EDTA, 0.14 M NaCl, pH 6.0 buffer. The sample was kept at 4° C. overnight and then it was desalted on a 5 ml Hi Trap™ column equilibrated with 0.1 M HEPES, pH 8.0 buffer.

Protein concentration was determined using BCA assay kit and the extent of luciferase labeling by biotin was estimated by HABA assay kit from Pierce.

The sample was stored at −80° C. in 0.1 M HEPES pH 8.0 buffer containing 1% casein and 5% glycerol.

TABLE 1
Comparison of in-solution (total activity) of biotinylated
luciferase relative and native luciferase.

| | | Signal intensity |
|---|---|---|
| Concentration of the | Signal intensity of native | of biotinylated |
| enzyme (10⁻⁶ g/mL) | luciferase (RLU × 10⁶) | luciferase (RLU × 10⁶) |
| 1.0 | 332 | 379 |
| 0.5 | 170 | 147 |
| 0.25 | 65 | 55 |
| 0.125 | 23 | 19 |
| 0.063 | 7 | 7 |

Samples were measured in duplicates and reported values are averages. Each sample contained 50 μL of either native or biotinylated luciferase and 50 μL of sample buffer (50 mM phosphate, pH 7.8 buffer containing 0.3 mM EDTA, 40 mM β-mercaptoethanol, 5% glycerol, 0.01% Tween-20 ™, 1 mM ATP and 0.5 mM D-luciferin). Bioluminescent reaction was initiated by adding 50 μl of 50 mM MgSO₄ prepared in distilled water. Luminescence was measured with 30 seconds integration time using Luminoskan Acent ® luminometer from Thermo Electron Corporation (Finland). Signal intensity was reported in Relative Light Units.

The specific activity of the biotinylated luciferase was assayed in a bioluminescent enzyme linked immunoassay as described below.

### Example 2

### Conjugation of Thermostable Firefly Luciferase to Antibody

### (a) Reversible protection of luciferase with 2,3-dimethylmaleic anhydride and activation of 2,3-dimethylmaleyl luciferase with 2-iminothiolane:

Luciferase (0.25 mg, 4.2×10⁻⁹ mol) was mixed with 0.27 mL 0.01 phosphate, 0.14 M NaCl buffer, pH 7.4.

DMMA (0.06 mg, 4.2×10⁻⁷ mol) that was prepared in anhydrous DMSO was added to the luciferase solution and the reaction proceeded for thirty minutes at 22° C. with constant, slow mixing.

After 30 minutes, the sample was centrifuged at 8000 g for fifteen minutes in concentrator tubes having molecular weight cut of value of 30 kDa, followed by washing with 0.01 M phosphate, 0.14 M NaCl, 5 mM EDTA buffer, pH 7.5 and centrifuged again for fifteen minutes at 8000 g.

The sample was reconstituted in 0.5 mL 0.01 M phosphate, 0.14 M NaCl, 5 mM EDTA pH 7.5 buffer. To the sample of 2,3-dimethylmaleyl luciferase, 2-imminotiolane (0.06 mg, 4.2×10⁻⁷ mol) prepared in the same buffer was added.

The activation reaction proceeded for 35 minutes at 22° C. with constant, slow mixing.

After activation, the sample was centrifuged twice at 8000 g using concentrator tubes with molecular weight cut off value of 30 kDa. After the final spin, the sample was reconstituted in 50 mM MES, 2 mM EDTA, 0.14 M NaCl buffer pH 6.0.

### (b) Activation of Mouse Anti-Human Creatine Kinase BB with sulfo-SMCC

Antibody (0.12 mg, 8×10⁻¹⁰ mol) in 0.1 M NaHCO₃ 0.2 M NaCl, 0.5 mM EDTA buffer pH 7.4 was mixed with sulfo-SMCC (0.02 mg, 3.6×10⁻⁸ mol) that was prepared in distilled water.

The activation reaction proceeded for 35 minutes at 22° C. and then the sample was centrifuged twice at 8000 g and washed with 0.01 M phosphate, 0.14 M NaCl, 5 mM EDTA buffer pH 7.5.

After the final spin, the sample was reconstituted in 50 mM MES, 2 mM EDTA, 0.14 M NaCl buffer pH 6.0.

### (c) Conjugation of 2-iminothiolane-activated 2,3-dimethylmaleyl luciferase with sulfo-SMCC activated antibody

Luciferase that has been chemically modified with 2,3-dimethylmaleic anhydride and then activated with 2-imonothiolane, as described above, was mixed with sulfo-SMCC-activated mouse anti-human creatine kinase BB antibody in 50 mM MES, 2 mM EDTA, 0.14 M NaCl buffer pH 6.0.

The conjugation reaction proceeded for 12 hours at 4° C.

Following the coupling reaction, the luciferase-antibody conjugate was purified by size exclusion chromatography on HiPrep 16/60 Sephacryl S200 HR column equilibrated with 50 mM MOPS, 0.3 M NaCl, 5 mM EDTA buffer pH 7.1.

The sample was stored at 4° C.

The activity of the luciferase conjugated to mouse anti-human creatine kinase BB antibody was assayed in a bioluminescent enzyme linked immunoassay as described below.

### Example 3

### Biochemical Immunoassay that Employs Biotinylated Firefly Luciferase

Greiner white strips were coated overnight with 4 μg/mL mouse anti-human troponin T M11.7 antibody in 0.1 M carbonate/bicarbonate buffer pH 9.6 at 4° C. and blocked with 1% casein for 60 minutes at 22° C. in 0.01 M phosphate, 0.14 M NaCl buffer (pH 7.4).

Antigen (troponin T) dilutions were prepared in human serum containing 5 mM EDTA and to each dilution was added mouse anti-human troponin T M7 antibody conjugated to streptavidin and biotinylated luciferase in 20 mM phosphate, 10 mM EDTA, 0.5 M NaCl, 0.5% Tween-20™ (polyoxyethylene (20) sorbitan monolaurate), 1% casein and 40 μg/mL mouse IgG buffer pH 7.4.

Reaction was incubated with vigorous shaking for 10 min at 22° C. After incubation, the strip wells were washed three times with 0.01 M phosphate, 0.25 M NaCl, 5 mM EDTA, 5 mM β-mercaptoethanol, 20 mM N-acetyl cysteine, 0.25% Tween-20™ buffer pH 7.4.

Following washing, to each strip well was added 50 μl of luciferase substrate buffer pH 7.8 containing 50 mM phosphate, 0.3 mM EDTA, 40 mM β-mercaptoethanol, 5% glycerol, 0.01% Tween-20™, 1 mM ATP and 0.5 mM D-luciferin.

A Bioluminescent reaction was initiated by injecting 50 μl of 50 mM MgSO₄ prepared in distilled water. Luminescence was measured with 30 seconds integration time.

TABLE 2
Light intensity generated during bioluminescent immunoassay
that utilized biotinylated luciferase.

| | Signal intensity of |
|---|---|
| Concentration of antigen Troponin T | biotinylated luciferase |
| M11.7 being detected (10⁻¹⁰ g/mL) | in a sandwich ELISA (RLU × 10⁴) |
| 1.87 | 66.4 |
| 0.94 | 31.8 |
| 0.47 | 17.2 |
| 0.23 | 9.7 |
| 0.12 | 6.5 |
| 0.06 | 4.7 |
| 0 | 3.0 |

Luminescence was measured with 30 seconds integration time using Luminoskan Acent ® luminometer from Thermo Electron Corporation (Finland).

### Example 4

### Biochemical Immunoassay that Employs the Firefly Luciferase Conjugated to Antibody

Greiner white strips were coated overnight with 5 μg/mL mouse anti-human creatine kinase MM antibody in 0.1 M carbonate/bicarbonate buffer pH 9.6 at 4° C. and then blocked with 1% casein for 60 minutes at 22° C. in 0.01 M phosphate, 0.14 M NaCl buffer pH 7.4.

Antigen (CKMB protein) dilutions were prepared in human serum and to each dilution was added mouse anti-human creatine kinase BB antibody conjugated to luciferase in 0.1 M MES, 0.5 M NaCl, 10 mM EDTA, 0.5% Tween-20™ (polyoxyethylene (20) sorbitan monolaurate), 0.1% pluronic acid F-68, 0.4% casein buffer pH 7.4.

Reaction was incubated with vigorous shaking for 15 min at 22° C. After incubation, the strip wells were washed three times with 0.01 M phosphate, 0.14 M NaCl, 5 mM EDTA, 5 mM β-mercaptoethanol, 0.25% Tween-20™ buffer pH 7.4.

Following washing, to each strip well was added 50 μl of luciferase substrate buffer pH 7.8 containing 50 mM phosphate, 0.5 mM EDTA, 40 mM β-mercaptoethanol, 5% glycerol, 0.01% Tween-20™, 1 mM ATP and 0.5 mM D-luciferin.

Bioluminescent reaction was initiated by injecting 50 μl of 50 mM MgSO₄ prepared in distilled water. Luminescence was measured with 30 seconds integration time.

TABLE 3
Light intensity generated during bioluminescent immunoassay
that utilized mouse anti-human creatine kinase BB antibody-luciferase
conjugate (antiCKBB-luciferase).

| | Signal intensity of |
|---|---|
| Concentration of antigen CKMB | antiCKBB-luciferase |
| protein being detected (10⁻⁷ g/mL) | in a sandwich ELISA (RLU × 10⁵) |
| 1.0 | 668 |
| 0.5 | 339 |
| 0.25 | 160 |
| 0.12 | 73 |
| 0.06 | 32 |
| 0 | 1.1 |
| Plate Background | 0.2 |

Luminescence was measured with 30 seconds integration time using Luminoskan Acent ® luminometer from Thermo Electron Corporation (Finland).

1. A method for covalent conjugation of luciferase with a chemical moiety comprising:
(a) reacting a luciferase with a reversible amino acid blocking or modifying agent to form an amino acid-modified luciferase;
(b) covalently coupling the amino acid-modified luciferase to the chemical moiety to form a covalently coupled, amino acid-modified luciferase;
(c) reversing the reaction of step (a) to form a covalently coupled luciferase.

2. A method as claimed in claim 1 wherein the reversible amino acid blocking or modifying agent is selected from the group consisting of (i) methylmaleic anhydride, (ii) 2,3-dimethylmaleic anhydride, (iii) dansylaminomethylmaleic anhydride, (iv) exo-cis-3,6-endo-epoxy-4,5-cis-epoxyhexahydrophthalic anhydride, (v) 3,4,5,6-tetrahydrophthalic anhydride, (vi) ninhydrin, and (vii) 2-aminothiophenol.

3. The method of claim 1 wherein the chemical moiety is selected from the group consisting of biotin, an antibody, a modified antibody, an antibody fragment, and a modified antibody fragment.

4. The method of claim 1 further comprising an additional step between step (a) and step (b), wherein such additional step comprises reacting the amino acid-modified luciferase with an amino acid blocking or modifying agent wherein such amino acid modifying agent modifies at least one amino acid not previously modified by the reversible amino acid modifying agent.

5. A method as claimed in claim 4 wherein the amino acid modifying agent is a covalent coupling agent.

6. A method as claimed in claim 5 wherein the amino acid blocking or modifying agent is selected from the group consisting of 2-iminothiolane, 1-ethyl-3-(3-dimethylaminopropyl)carbodiimide (EDC), N-hydroxysuccinimide (NHS) N-Hydroxysulfosuccinimide (Sulfo-NHS), Sulfosuccinimidyl acetate (Sulfo-NHS-Acetate), EZ-Link® Sulfo-NHS-Biotin, EZ-Link® Sulfo-NHS-LC-Biotin, EZ-Link® Sulfo-NHS-LC-LC-Biotin, EZ-Link® Sulfo-NHS-SS-Biotin, EZ-Link® NHS-PEO₄-Biotin, EZ-Link® NHS-Biotin, EZ-Link® NHS-LC-Biotin, EZ-Link® NHS-LC-LC-Biotin, EZ-Link® PFP-Biotin, EZ-Link® TFP-PEO-Biotin, EZ-Link® NHS-Iminobiotin-Trifluoroacetimaide, EZ-Link® Biotin-BMCC, EZ-Link® PEO-Iodoacetyl Biotin, EZ-Link® Iodoacetyl-LC-Biotin, EZ-Link® Biotinm-HPDP, EZ-Link® 5-(Biotinamido)pentylamine, EZ-Link® Biotin PEO-Amine, EZ-Link® Biotin-PEO-LC-Amine, EZ-Link® Biocytin-Hydrizide, EZ-Link® Biotin Hydrizide, EZ-Link® Biotin-LC-Hydrizide, EZ-Link® Psoralen-PEO-Biotin, EZ-Link® Photoactivatable Biotin, EZ-Link® Biotin-LC-ASA, EZ-Link® Biocytin, Bolton-Hunter reagent, Sufo-Bolton-Hunter reagent, Succinimidyl 4-hydrizinoic-otinate acetone hydrozone (SANH), C6-Succinimidyl 4-hydrazinoniconitate acetone hydrazone (C6-SANH), Succinimidyl 4-hydrazidoterephthalate hydrochloride (SHTH), Succinimidyl 4-formylbenzoate (SFB), C6-Succinimidyl 4-formylbenzoate (C6-SFB), N-5-Azido-2-nitrobenzoyloxysuccinimide (ANB-NOS), N-[4-(p-azidosalicylamido)butyl-3′-(2-pyridylthio)propionamide (APDP), p-Azidophenyl glyoxal monohydrate (APG), 4-(p-azidosaliclyamido)butilamine (ASBA), Bis-[β-(azidosalicylamido)ethyl]disulfide (BASED), 1,4-bis-maleimidobutane (BMB), 1,4-bis-maleimidyl-2,3-dihydroxybutane (BMDB), Bis-maleimidohexane (BMH), Bis-maleimidoethane (BMOE), N-β-Maleimidopropionic acid (BMPA), N-(β-Maleimidopropionic acid)hydrizide trifluoroacetic acid (BMPH), N-(β-Maleimidopropyloxy)succinimde ester (BMPS), 1,8-Bis-maleimidotriethyleneglycol (BMPEO₃), 1,11-Bis-maleimidotetraethylglycol (BMPEO₄), Bis[2-(succinimidyloxycarbonyloxy)ethyl]sulfone (BSOCOES), Bis(sulfosuccinimidyl)suberate, Dicyclohexylcarbodiimide (DCC), Dimethyl 3,3′-dithiobispropionimidate (DTBP), 3,3′-Dithiobis(sulfosuccinimidyl propionate) (DTSSP), 1,5-Diflouoro-2,4-dinitrobenzene (DFDNB), Dimethyl adipimidate hydrochloride salt (DMA), Dimethylpimelimidate hydrochloride salt (DMP), Dimethyl suberimate hydrochloride salt (DMS), 1,4-Di-[3′-(2′-pyridyldithio)propionamido]butane (DPDPB), Disuccinimidyl lutarate (DSg), Dithiobis(succinimidyl propionate) (DSP), Disuccinimidyl suberate (DSS), Disuccinimidyl tartrate (DST), Dithio-bis-maleimidomethane (DTME), Ethylene glycol bis(succinimidyl succinate) (EGS), N-ε-Maleimidocaproic acid (EMCA), N-ε-maleimidocaproic acid hydrizide (EMCH), N-(ε-Maleimidocarpyloxy)succinimide ester (EMCS), N-(γ-Maleimidobuturyloxy)succinimide ester (GMBS), 1,6-Hexane-bis-vinylsulfone (HBVS), N-κ-Maleimidoundecanoic acid (KMUA), N-κ-Maleimidoundecanoic acid hydrizide (KMUH), Succinimidyl-4-(N-maleimidomethyl)cyclohexane-1-carboxy(6-amidocaproate) (LC-SMCC), Succinimidyl 6-[3-(2-pyridyldithio)propionamido]hexanoate (LC-SPDP), m-Melaimidobenzoyl-Nhydroxy succinimide (MBS), 4-(4-N-maleimidophenyl)butyric acid (MPBH), Methyl N-succinimidyl adipate (MSA), N-hyroxysuccinimidyl-4-azidosalicylic acid (NHS-ASA), 3-(2-Pyridyldithio)propionul hydrazide (PDPH), N-(p-maleimidophenyl)isocyanate (PMPI), N-succinimidyl (4-azidophenyl) 1,3′-dithiopropionate (SADP), sulfosuccinimidyl 2-(7-azido-4-methyl-coumarin-3-acetamido)ethyl-1,3′-dithiopropionate (SAED), Sulfosuccinimidyl 2-(m-azido-o-nitrobenzamido)ethyl-1,3′-dithiopropionate (SAND), N-Succinimidyl 6-(4′-azido-2′-nitrophenylamino)hexanoate (SANPAH), Sulfosuccinimidyl-2-(-p-azido-salicylamido)ethyl-1,3′-dithiopropionate (SASD), N-Succinimidyl S-acetylthioacetate (SATA), N-succinimidyl S-acetylthiopropionate (SATP), Succinimidyl 3-(bromoacetamido)propionate (SBAP), Sulfosuccinimidyl-(perfluoroazidobenzamido)ethyl-1,3′-dithiopropionate (SFAD), N-Succinimidyl iodoacetate (SIA), N-Succinimidyl(4-iodoacetyl)aminobenzoate (SIAB), Succinimidyl 4-(N-maleimidomethyl)cyclohexane-1-carboxylate (SMCC), Succinimidyl 4-(p-maleimidophenyl)butyrate (SMPB), Succinimidyl-6-(β-maleimidopropionamido)hexanoate (SMPH), 4-Succinimidyloxycarbonyl-methyl-α-(2-propyldithio)toluene (SMPT), Succinimidyl-[4-(psoralen-8-yloxy)butyrate (SPB), N-Succinimidyl 3-(2-pyridylthio)propionate (SPDP), Disulfosuccinimidyl tartrate (Sulfo-DST), Ethylene glycol bis(sulfosuccinimidyl succinate) (Sulfo-EGS), N-(ε-Maleimidocapryloxy)sulfosuccinimide ester (Sulfo-EMCS), N-(γ-Maleimidobuturyloxy)sulfosuccinimide ester (Sulfo-GMBS), N-Hydroxysulfosuccinimidyl-4-azidobenzoate (Sulfo-HSAB), N-(κ-Maleimidoundecanoyloxy)sulfosuccinimide ester (Sulfo-KMUS), Sulfosuccinimidyl-6-[α-methyl-α-(2-pyridyldithio)toluamido]hexanoate (Sulfo-LC-SMPT), Sulfosuccinimidyl 6-[3′-(2-pyridyldithio)propionamido]hexanoate (Sulfo-LC-SPDP), m-Maleimidobenzoyl-N-hydroxysulfosuccinimide (Sulfo-MBS), Sulfosuccinimidyl(4-azidosalicylamido)hexanoate (Sulfo-NHS-LC-ASA), Sulfosuccinimidyl(4-azidophenyldithio)propionate (Sulfo-SADP), Sulfo-succinimidyl 6-(4′-azido-2′-nitro-phenylamino)hexanoate (Sulfo-SANPAH), Sulfosuccinimidyl(4-iodoacetyl)aminobenzoate (Sulfo-SIAB), Sulfosuccinimidyl 4-(N-maleimidomethyl)cyclohexane-1-carboxylate (Sulfo-SMCC), Sulfosuccinimidyl 4-(p-maleimidophenyl)butyrate (Sulfo-SMPB), N-(ε-Trifluoroacetylcapryloxy)succinimide ester (TFCS), Sulfosuccinimidyl[2-6-(biotinamido)-2-(p-azidobenzamido)-hexanoamido[ethyl-1,3′-dithiopropionate (Sulfo-SBED), Succinimidyl[2-6-(biotinamido)-2-(p-azidobenzamido)-hexanoamido[ethyl-1,3′-dithiopropionate (SBED), Tris-succinimidyl aminotriacetate (TSAT), β-[Tris(hydroxymethyl)phosphino]propionic acid (THPP), Tris(2-maleimidomethyl)amine (TMEA), N-(Iodoethyl)trifluoroacetamide, 2-Aminoethyl-2′-aminothanethiosulfonate, 5,5′-Dithio-bis(2-nitrobenzoic acid), N-Ethylmaleimide, p-Hydroxyphenylglyoxal, Iodoacetic acid, Phenylmethylsulfonyl fluoride (PMSF), 1-Fluoro-2,4-dinitrophenyl-5-L-alenine amide (FDAA), Fluoraldehyde o-Phthaldehyde, Ninhydrin, Phenylisothiocyanate, Ammonium 4-chloro-7-sulfobenzo-furzan (SBF-Chloride), 2,4,6-Trinitrobenzene sulfonic acid, and Methylmethanothiosulfonate (MMTS).

7. The method of claim 1 further comprising: (1) a blocking step prior to step (a), wherein such blocking step comprises reacting the luciferase with a luciferin substrate or an Mg-ATP complex substrate; and (2) a washing step prior to, during or after step (c) wherein such washing step removes the luciferin substrate or the Mg-ATP complex substrate.

8. The method of claim 1 wherein step (a) occurs at a pH of between 5 and 9.

9. The method of claim 1 wherein step (a) occurs for between 10 minutes and 24 hours.

10. The method of claim 9 wherein step (a) occurs for between 30 and 60 minutes.

11. The method of claim 1 wherein the ratio of reversible amino acid modifying agent to luciferase in step (a) is 80 to 100 molar equivalents of reversible amino acid modifying agent to luciferase.

12. The method of claim 1 wherein the ratio of chemical moiety to amino acid-modified luciferase in step (b) is between 0.2-10 molar equivalents of chemical moiety to amino acid-modified luciferase.

13. The method of claim 1 wherein step (b) occurs at a pH of between 6 and 9.

14. The method of claim 1 wherein the covalent coupling in step (b) is done by mixing.

15. The method of claim 4 wherein the ratio of amino acid modifying agent to amino acid-modified luciferase in the additional step is about 100 molar equivalents of amino acid modifying agent to amino acid-modified luciferase.

16. The method of claim 1 wherein the reversal step (c) comprises hydrolysis of the amino acid-modified luciferase.

17. The method of claim 1 wherein steps (b) and (c) occur simultaneously.

18. The method of claim 1 wherein the reversal step (c) occurs at a pH of about 6.

19. A method for performing a binding assay comprising use of a covalently conjugated luciferase made by a method of claim 1.

20.-33. (canceled)

34. A reversibly chemically modified luciferase made by a method comprising reacting luciferase with a reversible amino acid modifying agent to form an amino-acid modified luciferase.
